(19) United States
(12) Patent Application Publication
(10) Pub. No.: US 2017/0346071 A1
(43) Pub. Date: Nov. 30, 2017

(54) NONAQUEOUS ELECTROLYTE SECONDARY BATTERY

(71) Applicant: Sanyo Electric Co., Ltd., Daito-shi (JP)

(72) Inventors: Atsushi Ogata, Osaka (JP); Katsunori Yanagida, Hyogo (JP); Takanobu Chiga, Osaka (JP); Naoya Morisawa, Hyogo (JP); Yoshiyuki Muraoka, Osaka (JP)

(73) Assignee: Sanyo Electric Co., Ltd., Daito-shi (JP)

(21) Appl. No.: 15/678,340

(22) Filed: Aug. 16, 2017

Related U.S. Application Data
(63) Continuation of application No. PCT/JP2016/000266, filed on Jan. 20, 2016.

(30) Foreign Application Priority Data
Mar. 26, 2015 (JP) 2015-064920

(51) Int. Cl. H01M 4/131; H01M 10/0525; C07C 69/63; C07D 317/42

(52) U.S. Cl. CPC H01M 4/131; H01M 10/0525; C07C 69/63; C07D 317/42

(57) ABSTRACT

Nonaqueous electrolyte secondary batteries are provided which are resistant to a decrease in capacity associated with charge and discharge cycles and have excellent discharge rate characteristics. A nonaqueous electrolyte secondary battery according to an example embodiment includes a positive electrode including a lithium transition metal oxide, a negative electrode and a nonaqueous electrolyte. The lithium transition metal oxide has a content of voids within particles of 0.2 to 30% before first charging. The nonaqueous electrolyte includes a fluorinated cyclic carbonate and a fluorinated chain carboxylate ester.

### TECHNICAL FIELD

The present disclosure relates to nonaqueous electrolyte secondary batteries.

### BACKGROUND ART

To enhance output characteristics, Patent Literature 1 presents a nonaqueous electrolyte secondary battery which includes a positive electrode active material having a content of voids within particles of 3 to 30%. Some preferred solvents in the nonaqueous electrolyte described in Patent Literature 1 are carbonate ester solvents such as ethylene carbonate (EC), diethyl carbonate (DEC), dimethyl carbonate (DMC) and propylene carbonate (PC), and γ-butyrolactone, tetrahydrofuran and acetonitrile.

### CITATION LIST

### Patent Literature

PTL 1: International Publication No. WO 2012/137391

### SUMMARY OF INVENTION

### Technical Problem

In nonaqueous electrolyte secondary batteries, the suppression of a decrease in capacity associated with charge and discharge cycles is an important problem to be solved. Further, a high discharge capacity during high-rate discharging is desired. Even the technique disclosed in Patent Literature 1 cannot solve these problems sufficiently and still has plenty of room for improvement.

### Solution to Problem

A nonaqueous electrolyte secondary battery according to one aspect of the present disclosure includes a positive electrode including a lithium transition metal oxide, a negative electrode and a nonaqueous electrolyte, the lithium transition metal oxide having a content of voids within particles of 0.2 to 30% before first charging, the nonaqueous electrolyte including a fluorinated cyclic carbonate and a fluorinated chain carboxylate ester.

### Advantageous Effects of Invention

The nonaqueous electrolyte secondary battery according to one aspect of the present disclosure is resistant to a decrease in capacity associated with charge and discharge cycles and has excellent discharge rate characteristics.

### BRIEF DESCRIPTION OF DRAWINGS

FIG. 1 is a sectional view of a nonaqueous electrolyte secondary battery according to an example embodiment.

FIG. 2 is a sectional electron micrograph of a particle, before charge and discharge cycles, of a positive electrode active material (EXAMPLE 1) constituting a nonaqueous electrolyte secondary battery according to an example embodiment.

FIG. 3 is a sectional electron micrograph of a particle, after charge and discharge cycles, of the positive electrode active material (EXAMPLE 1) constituting the nonaqueous electrolyte secondary battery according to an example embodiment.

FIG. 4 is a sectional electron micrograph of a particle, after charge and discharge cycles, of a positive electrode active material (COMPARATIVE EXAMPLE 1) constituting a conventional nonaqueous electrolyte secondary battery.

FIG. 5 is a sectional electron micrograph of a particle, before charge and discharge cycles, of a positive electrode active material (COMPARATIVE EXAMPLE 2) constituting a conventional nonaqueous electrolyte secondary battery.

### DESCRIPTION OF EMBODIMENTS

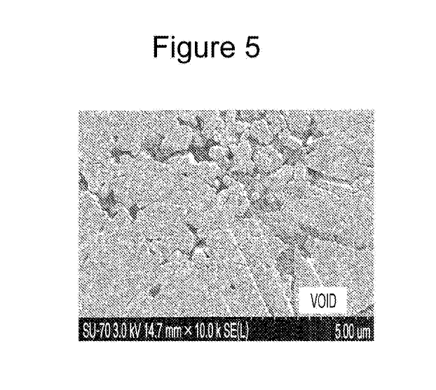

General positive electrode active materials used in nonaqueous electrolyte secondary batteries have little voids in the particles, and the void content is less than 0.2% (see FIG. 5). The present inventors have found that cycle characteristics and discharge rate characteristics are specifically improved by using, as the positive electrode active material, a lithium transition metal oxide having a content of voids within particles of 0.2 to 30% before the first charging, and, as the solvent in the nonaqueous electrolyte, a fluorinated cyclic carbonate and a fluorinated chain carboxylate ester. Studies conducted by the present inventors have shown that the introduction of voids to a positive electrode active material results in a decrease in battery characteristics if the nonaqueous electrolyte secondary battery is free from fluorinated cyclic carbonates and fluorinated chain carboxylate esters (see COMPARATIVE EXAMPLE 1).

Hereinbelow, an example embodiment will be described in detail.

The drawings used in the description of the embodiment are schematic, and the configurations of the constituents illustrated in the drawings such as sizes are sometimes different from the actual ones. Specific configurations such as sizes should be estimated in consideration of the description given below.

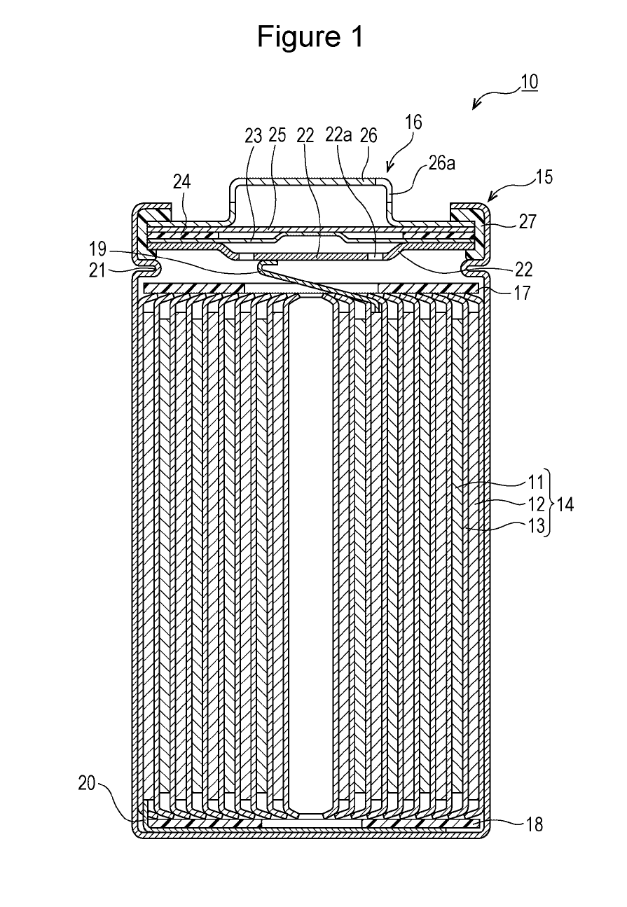

FIG. 1 is a sectional view of a nonaqueous electrolyte secondary battery 10 according to an example embodiment.

The nonaqueous electrolyte secondary battery 10 includes a positive electrode 11, a negative electrode 12 and a nonaqueous electrolyte. Preferably, a separator 13 is disposed between the positive electrode 11 and the negative electrode 12. For example, the nonaqueous electrolyte secondary battery 10 has a structure in which a wound electrode assembly 14 that includes the positive electrode 11 and the negative electrode 12 wound together via the separator 13, and the nonaqueous electrolyte are accommodated in a battery case. The wound electrode assembly 14 may be replaced by other form of an electrode assembly such as a stacked electrode assembly which includes positive electrodes and negative electrodes stacked alternately on top of one another via separators. Examples of the battery cases for accommodating the electrode assembly 14 and the nonaqueous electrolyte include metallic cases such as cylindrical cases, prismatic cases, coin-shaped cases and button-shaped cases, and resin cases formed by laminating resin sheets (laminate batteries). In the example illustrated in FIG. 1, the battery case is composed of a bottomed cylindrical case body 15 and a sealing body 16.

The nonaqueous electrolyte secondary battery 10 includes insulating plates 17 and 18 disposed on and under the electrode assembly 14. In the example illustrated in FIG. 1, a positive electrode lead 19 attached to the positive electrode 11 extends toward the sealing body 16 through an opening in the insulating plate 17, and a negative electrode lead 20 attached to the negative electrode 12 extends on the outside of the insulating plate 18 along the bottom of the case body 15. For example, the positive electrode lead 19 is connected to the lower surface of a filter 22 that is a bottom plate of the sealing body 16 by a technique such as welding, and a cap 26 that is a top plate of the sealing body 16 is electrically connected to the filter 22 and serves as a positive electrode terminal. The negative electrode lead 20 is connected to the inner bottom surface of the case body 15 by a technique such as welding, and the case body 15 serves as a negative electrode terminal. In the present embodiment, a current interrupt device (CID) and a degassing mechanism (a safety valve) are disposed in the sealing body 16. Preferably, a degassing valve is disposed also in the bottom of the case body 15.

The case body 15 is, for example, a bottomed cylindrical container made of a metal. A gasket 27 is disposed between the case body 15 and the sealing body 16, thereby ensuring airtightness inside the battery case. The case body 15 preferably has a protrudent portion 21 which is formed by, for example, pressing a lateral portion by a force applied from the outer side and which supports the sealing body 16. The protrudent portion 21 is preferably famed as a circle along the peripheral direction of the case body 15, and supports the sealing body 16 on its upper surface.

The sealing body 16 has a filter 22 with a filter opening 22a, and a valve disposed on the filter 22. The valve covers the filter opening 22a of the filter 22, and is broken in the event of an increase in the inside pressure of the battery by heat generated due to abnormalities such as internal short-circuits. In the present embodiment, the valve includes a lower valve 23 and an upper valve 25; in addition, an insulating member 24 between the lower valve 23 and the upper valve 25, and a cap 26 having a cap opening 26a are further disposed. For example, the members constituting the sealing body 16 have a disk shape or a ring shape, and the members except the insulating member 24 are electrically connected to one another. Specifically, the filter 22 and the lower valve 23 are joined together at their peripheral portions, and the upper valve 25 and the cap 26 are also joined together at their peripheral portions. The lower valve 23 and the upper valve 25 are connected to each other at their central portions, with the insulating member 24 being disposed between the peripheral portions of the valves. If the inside pressure is elevated by heat due to an abnormality such as internal short-circuit, for example, a thin portion of the lower valve 23 is broken to cause the upper valve 25 to swell toward the cap 26 away from the lower valve 23, thereby interrupting the electrical connection between the valves.

### [Positive Electrodes]

For example, the positive electrode is composed of a positive electrode current collector such as a metallic foil, and a positive electrode mixture layer disposed on the positive electrode current collector. Examples of the positive electrode current collectors include foils of metals that are stable at positive electrode potentials such as aluminum, and films having a skin layer of such a metal. The positive electrode mixture layer includes a lithium transition metal oxide, and preferably further includes a conductive agent and a binder. The lithium transition metal oxide functions as a positive electrode active material. The positive electrode active material may be a single lithium transition metal oxide or a combination of two or more kinds of such oxides. In the present embodiment, the positive electrode active material that is used is a lithium transition metal oxide alone (the positive electrode active material is the lithium transition metal oxide itself). The positive electrode may be fabricated by, for example, applying a positive electrode mixture slurry including the positive electrode active material and other components such as a conductive agent and a binder onto a positive electrode current collector, and drying and rolling the wet films so as to form positive electrode mixture layers on both sides of the current collector.

The conductive agent may be used to enhance the electrical conductivity of the positive electrode mixture layers. Examples of the conductive agents include carbon materials such as carbon black, acetylene black, Ketjen black and graphite. These may be used singly, or two or more may be used in combination.

The binder may be used to enhance the bonding of components such as the positive electrode active material to the surface of the positive electrode current collector while ensuring a good contact between the positive electrode active material and the conductive agent. Examples of the binders include fluororesins such as polytetrafluoroethylene (PTFE) and polyvinylidene fluoride (PVdF), polyacrylonitrile (PAN), polyimide resins, acrylic resins and polyolefin resins. These resins may be used in combination with carboxymethylcellulose (CMC) or salts thereof (such as CMC-Na, CMC-K and CMC-NH₄, and partially neutralized salts), polyethylene oxide (PEO) and the like. These may be used singly, or two or more may be used in combination.

The proportions of the positive electrode active material, the conductive agent and the binder are individually preferably in the range of 80 to 98 mass % positive electrode active material, 0.8 to 20 mass % conductive agent, and 0.8 to 20 mass % binder. These proportions ensure that a high energy density and good cycle characteristics will be obtained. If the proportion of the positive electrode active material exceeds 99 mass %, the electron conductivity within the positive electrode is decreased, and the capacity may be decreased and the cycle characteristics may be deteriorated due to heterogeneous reaction at times.

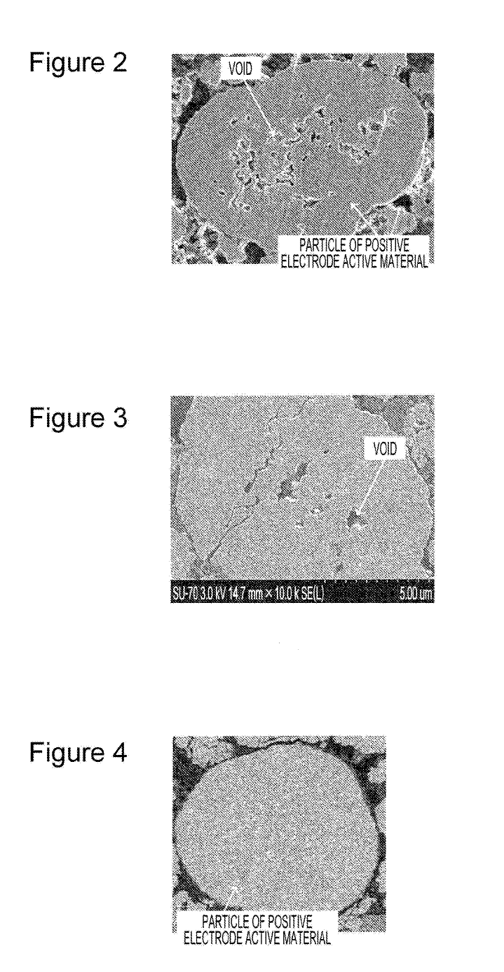

Hereinbelow, a positive electrode active material (lithium transition metal oxide) representing an example embodiment will be described in detail. FIGS. 2 and 3 are scanning electron micrographs (SEM images) of a cross section of a particle famed with a cross section polisher (CP) (hereinafter, written as “CP cross section”) of the lithium transition metal oxide representing an example embodiment. FIG. 2 is a SEM image before charge and discharge cycles, and FIG. 3 is a SEM image after 400 cycles.

Examples of the lithium transition metal oxide (hereinafter, written as the “composite oxide A”) include those composite oxides containing such a transition metal element as Co, Mn or Ni. For example, the composite oxide A is LiₓCoO₂, LiₓNiO₂, LiₓMnO₂, LiₓCo_{y}Ni_{1-y}O₂, LiₓCo_{y}M_{1-y}O_{z}, LiₓNi_{1-y} M_{y}O_{z}, LiₓMn₂O₄, LiₓMn_{2-y}M_{y}O₄, LiMPO₄ or Li₂MPO₄F (M: at least one of Na, Mg, Sc, Y, Mn, Fe, Co, Ni, Cu, Zn, Al, Cr, Pb, Sb and B, 0<x≦1.2, 0<y≦0.9, and 2.0≦z≦2.3).

A preferred example of the composite oxides A is a composite oxide containing more than 30 mol % Ni relative to the total number of moles of the metal elements except Li. From points of view such as low cost and high capacity, the Ni content is preferably higher than 30 mol %. For example, the composite oxide A is an oxide represented by the general formula LiₐCoₓNi_{y}M_{(1-x-y)}O₂ {0.1≦a≦1.2, 0<x<0.4, 0.3<y<1, 0.3<x+y<1, M: at least one of Na, Mg, Sc, Y, Mn, Fe, Co, Ni, Cu, Zn, Al, Cr, Pb, Sb and B, preferably at least one selected from Mn, Al and Zr} and having a layered rock-salt crystal structure.

The composite oxide A is secondary particles formed by the aggregation of many primary particles. Thus, the composite oxide A has grain boundaries formed by primary particles. Although the secondary particles are aggregated at times, such aggregated secondary particles can be separated from one another by ultrasonic dispersion. The volume average particle size (hereinafter, written as “Dv”) of the composite oxide A is preferably 7 to 30 μm, more preferably 8 to 30 μm, and particularly preferably 9 to 25 μm. The Dv is the particle size (median size) at 50% cumulative volume in the particle size distribution, and may be measured by a light diffraction scattering method.

The average crystallite size of the composite oxide A is preferably 40 to 140 nm, and more preferably 40 to 100 nm. When the average crystallite size is in this range, the active material is swollen and shrunk during initial charging in an equalized manner, and cycle characteristics are further enhanced. If the crystallite size of the composite oxide A exceeds 140 nm, the swelling and shrinkage of the crystal in a specific direction, in particular, in the c axis direction during charging and discharging sometimes causes a breakage of a quality film, present on the surface of the oxide particles, that suppresses the side reaction of the oxide with the electrolytic solution. As a result, the current is concentrated to regions in which there is little deposition of such a film and the electron resistance is low, giving rise to a risk that the active material is degraded and cycle characteristics are deteriorated. If, on the other hand, the crystallite size is smaller than 40 nm, the growth of the crystal is so insufficient that the intercalation and deintercalation of lithium ions are inhibited and the capacity of the positive electrode is decreased at times. The average crystallite size is measured by the method described in EXAMPLES later.

As illustrated in FIGS. 2 and 3, the composite oxide A has a large number of voids inside the particle. The voids are spaces famed among primary particles that constitute a secondary particle of the composite oxide A, and they increase the surface area of the composite oxide A that contributes to the battery reaction, that is, the field of the reaction with the electrolytic solution. Specifically, the voids present inside the composite oxide A (a secondary particle) have, for example, partial connections to one another and are open to the surface of the secondary particle to allow an entry of the electrolytic solution. However, all the voids do not need to be open to the surface of the secondary particle and some voids may be closed to the entry of the electrolytic solution.

The composite oxide A has a content of voids within particles of 0.2 to 30% before the first charging. The void content is preferably 0.5 to 20%, and more preferably 2 to 15%. When the void content is in this range, a distortion that is produced between particles by the swelling and shrinkage of the active material during charging and discharging may be relaxed and thereby the particles can be prevented from breakage; further, the oxide has an increased area of the field of the reaction with the electrolytic solution. In addition, the above void content ensures that a quality protective film will be formed over a large area of the oxide surface including the insides of the voids by virtue of the synergetic effect with solvent components of the electrolytic solution described later. If the void content is less than 0.2%, the area of the field of the reaction with the electrolytic solution is so small that the concentration of the current to the electrochemically active surface causes poorly ion conductive products, resulting from the decomposition of the electrolytic solution, to be deposited in a large thickness to cause a decrease in discharge capacity; further, the particles fail to relax the distortion due to a volume change of the active material during charging and discharging, and consequently the active material particles are broken and the capacity retention is decreased. If, on the other hand, the void content is above 30%, the discharge capacity per unit volume of the active material is disadvantageously decreased.

The void content of the composite oxide A means the proportion of the area occupied by voids relative to the total area of a cross section of the oxide particle, and may be determined by SEM observation of cross sections of the particles. A specific method for the measurement of the void content is described below.

(1) A CP cross section of the composite oxide A is obtained. This process may involve, for example, a cross section polisher (ex. SM-09010) manufactured by JEOL Ltd.

(2) The CP cross section obtained (the cross section of the particle exposed) is observed by SEM, and the outline of the particle is drawn. (3) The proportion of the area of voids present in the region enclosed by the outline is measured relative to the total area of the region enclosed by the outline (the total area of the CP cross section), and the void content is calculated by (area of voids/total area of CP cross section)×100. The void content is the average of the measurement results of 100 particles.

The void content of the composite oxide A does not change greatly even after repeated cycles of charging and discharging (see FIG. 3). If a breakage occurs in the particles during charging and discharging, the void content is significantly increased (see FIG. 4). In other words, the nonaqueous electrolyte secondary battery of the present embodiment is resistant to the breakage of the particles of the positive electrode active material (the composite oxide A) that is associated with charging and discharging, and the void content after initial cycles, for example, after less than 100 cycles is substantially the same as the void content before the first charging. The void content of the composite oxide A, even after 400 cycles, is preferably not more than 30% and is, for example, 0.2 to 20% or 0.5 to 15%.

The void content of the composite oxide A may be manipulated by controlling conditions such as the ratio in which a lithium compound and a transition metal compound are mixed, the type of a precursor, and the temperature, time and atmosphere during calcination. When, for example, a lithium raw material and a transition metal compound are mixed together with a lithium/transition metal molar ratio exceeding 1.2, the amount of voids is decreased with the progress of sintering during the calcination. If the lithium/transition metal molar ratio is 0.9 or less, the proportion of the compound that does not contribute to charging and discharging is increased and consequently the capacity may be decreased. Calcination at a high temperature allows sintering to proceed to a further extent, and consequently the void content tends to be low. The calcination time and atmosphere are similar important factors. Decreasing the calcination temperature increases the amount of voids but also works against the progress of the reaction between the lithium compound and the transition metal compound, sometimes resulting in an increase in the proportion of unreacted compounds.

### [Negative Electrodes]

For example, the negative electrode is composed of a negative electrode current collector such as a metallic foil, and a negative electrode mixture layer disposed on the current collector. Examples of the negative electrode current collectors include foils of metals that are stable at negative electrode potentials such as copper, and films having a skin layer of such a metal. The negative electrode mixture layer includes a negative electrode active material, and preferably further includes a binder. The negative electrode may be fabricated by, for example, applying a negative electrode mixture slurry including the negative electrode active material and other components such as a binder onto a negative electrode current collector, and drying and rolling the wet films so as to form negative electrode mixture layers on both sides of the current collector.

The negative electrode active material is not particularly limited as long as it can reversibly store and release lithium ions. Examples include carbon materials such as natural graphite and artificial graphite, metals which can be alloyed with lithium such as silicon (Si) and tin (Sn), and alloys and composite oxides containing metal elements such as Si and Sn. The negative electrode active materials may be used singly, or two or more may be used in combination.

Examples of the binders include, similarly to those in the positive electrodes, fluororesins, PAN, polyimide resins, acrylic resins and polyolefin resins. When the mixture slurry is prepared using an aqueous solvent, it is preferable to use CMC or a salt thereof (such as CMC-Na, CMC-K or CMC-NH₄, or a partially neutralized salt), styrene-butadiene rubber (SBR), polyacrylic acid (PAA) or a salt thereof (such as PAA-Na or PAA-K, or a partially neutralized salt), polyvinyl alcohol (PVA) or the like.

The proportion of the negative electrode active material and that of the binder are individually desirably in the range of 93 to 99 mass % negative electrode active material, and 0.5 to 10 mass % binder. These proportions ensure that a high energy density and good cycle characteristics will be obtained.

### [Separators]

As the separators, porous sheets having ion permeability and insulating properties are used. Specific examples of the porous sheets include microporous thin films, woven fabrics and nonwoven fabrics. Some preferred materials of the separators are polyolefin resins such as polyethylene and polypropylene, and celluloses. The separators may be stacks having a cellulose fiber layer and a thermoplastic resin fiber layer such as of a polyolefin resin. Alternatively, the separators may be multilayered separators including a polyethylene layer and a polypropylene layer, or may be separators having a coating of an aramid resin or the like on the surface.

A filler layer including an inorganic filler may be disposed in the interface of the separator and at least one of the positive electrode and the negative electrode. Examples of the inorganic fillers include oxides containing at least one of titanium (Ti), aluminum (Al), silicon (Si) and magnesium (Mg), and phosphoric acid compounds. For example, the filler layer may be formed by applying a slurry containing the filler onto the surface of the positive electrode, the negative electrode or the separator.

### [Nonaqueous Electrolytes]

The nonaqueous electrolyte includes a nonaqueous solvent and an electrolyte salt dissolved in the nonaqueous solvent. The nonaqueous solvent includes at least a fluorinated cyclic carbonate and a fluorinated chain carboxylate ester. By virtue of the nonaqueous electrolyte containing a fluorinated cyclic carbonate and a fluorinated chain carboxylate ester, a quality film is formed on the surface of the positive electrode active material particles having voids, and suppresses the deposition of byproducts. The proportion of the fluorinated cyclic carbonate and the fluorinated chain carboxylate ester relative to the total volume of the nonaqueous solvent is preferably not less than 50 vol %. Preferably, the solvent includes the fluorinated chain carboxylate ester in a higher proportion than the fluorinated cyclic carbonate.

Examples of the fluorinated cyclic carbonates include 4-fluoroethylene carbonate (FEC), 4,5-difluoro-1,3-dioxolan-2-one, 4,4-difluoro-1,3-dioxolan-2-one, 4-fluoro-5-methyl-1,3-dioxolan-2-one, 4-fluoro-4-methyl-1,3-dioxolan-2-one, 4-trifluoromethyl-1,3-dioxolan-2-one and 4,5-difluoro-4,5-dimethyl-1,3-dioxolan-2-one (DFBC). Of these, FEC is particularly preferable. The content of the fluorinated cyclic carbonate is preferably 2 to 40 vol %, and more preferably 5 to 30 vol %. If the content of the fluorinated cyclic carbonate is less than 2 vol %, a sufficient film is not formed on the surface of the positive electrode active material and the increase in the resistance of the positive electrode active material after long-term cycles cannot be prevented at times. If the content of the fluorinated cyclic carbonate exceeds 40 vol %, an excessively large amount of gas may be generated by the decomposition of the electrolytic solution.

Examples of the fluorinated chain carboxylate esters include carboxylate esters such as methyl acetate, ethyl acetate, propyl acetate, methyl propionate and ethyl propionate which are partially fluorinated in place of a hydrogen atom. Of these, fluoro methyl propionate (FMP) is particularly preferable. The FMP is preferably methyl 3,3,3-trifluoropropionate. The content of the fluorinated chain carboxylate ester is preferably 20 to 95 vol %, and more preferably 30 to 90 vol %. If the content of the fluorinated chain carboxylate ester is less than 20 vol %, a sufficient film is not formed on the surface of the positive electrode active material and the increase in the resistance of the positive electrode active material after long-team cycles cannot be prevented at times. If the content of the fluorinated chain carboxylate ester exceeds 95 vol %, the electrical conductivity of the electrolytic solution is disadvantageously decreased.

The nonaqueous solvent may include a fluorine-containing solvent other than FEC and FMP, for example, a lower chain carbonate ester such as dimethyl carbonate, ethyl methyl carbonate, diethyl carbonate, methyl propyl carbonate, ethyl propyl carbonate or methyl isopropyl carbonate which is partially fluorinated in place of a hydrogen atom.

The nonaqueous solvent may include a fluorine-free solvent. Examples of the fluorine-free solvents include cyclic carbonates, chain carbonates, carboxylate esters, cyclic ethers, chain ethers, nitriles such as acetonitrile, amides such as dimethylformamide, and mixed solvents of these solvents.

Examples of the cyclic carbonates include ethylene carbonate (EC), propylene carbonate (PC) and butylene carbonate. Examples of the chain carbonates include dimethyl carbonate, methyl ethyl carbonate (EMC), diethyl carbonate, methyl propyl carbonate, ethyl propyl carbonate and methyl isopropyl carbonate.

Examples of the carboxylate esters include methyl acetate, ethyl acetate, propyl acetate, methyl propionate (MP), ethyl propionate and γ-butyrolactone.

Examples of the cyclic ethers include 1,3-dioxolane, 4-methyl-1,3-dioxolane, tetrahydrofuran, 2-methyltetrahydrofuran, propylene oxide, 1,2-butylene oxide, 1,3-dioxane, 1,4-dioxane, 1,3,5-trioxane, furan, 2-methylfuran, 1,8-cineole and crown ethers.

Examples of the chain ethers include 1,2-dimethoxyethane, diethyl ether, dipropyl ether, diisopropyl ether, dibutyl ether, dihexyl ether, ethyl vinyl ether, butyl vinyl ether, methyl phenyl ether, ethyl phenyl ether, butyl phenyl ether, pentyl phenyl ether, methoxytoluene, benzyl ethyl ether, diphenyl ether, dibenzyl ether, o-dimethoxybenzene, 1,2-diethoxyethane, 1,2-dibutoxyethane, diethylene glycol dimethyl ether, diethylene glycol diethyl ether, diethylene glycol dibutyl ether, 1,1-dimethoxymethane, 1,1-diethoxyethane, triethylene glycol dimethyl ether and tetraethylene glycol dimethyl ether.

The electrolyte salt is preferably a lithium salt. Examples of the lithium salts include LiBF₄, LiClO₄, LiPF₆, LiAsF₆, LiSbF₆, LiAlCl₄, LiSCN, LiCF₃SO₃, LiCF₃CO₂, Li(P(C₂O₄)F₄), LiPF₆₋ₓ(CₙF₂ₙ₊₁)ₓ (1<x<6, n is 1 or 2), LiB₁₀Cl₁₀, LiCl, LiBr, LiI, chloroborane lithium, lithium lower aliphatic carboxylates, borate salts such as Li₂B₄O₇ and Li(B(C₂O₄)F₂), and imide salts such as LiN(SO₂CF₃)₂ and LiN(CₗF₂ₗ₊₁SO₂)(CₘF₂ₘ₊₁SO₂) {l and m are integers of 1 or greater}. The lithium salt may be a single salt or a mixture of a plurality of salts. Of these, from points of view such as ion conductivity and electrochemical stability, LiPF₆ is preferably used. The concentration of the lithium salt is preferably 0.8 to 1.8 mol per 1 L of the nonaqueous solvent.

The nonaqueous electrolyte may contain a sultone compound such as 1,3-propanesultone (PS) or 1,3-propenesultone (PRS), 1,6-hexamethylene diisocyanate (HDMI), vinylene carbonate (VC), pimelonitrile (PN) or the like.

### EXAMPLES

Hereinbelow, the present disclosure will be described in greater detail based on EXAMPLES. The scope of the present disclosure is not limited to such EXAMPLES.

### Example 1

### [Preparation of Lithium Transition Metal Composite Oxide (Positive Electrode Active Material)]

In a reaction vessel, an aqueous solution was provided which contained cobalt ions, nickel ions and manganese ions derived from cobalt sulfate, nickel sulfate and manganese sulfate. The molar ratio of cobalt, nickel and manganese (nickel:cobalt:manganese) in the aqueous solution was adjusted to 5:2:3. Next, an aqueous sodium hydroxide solution was added dropwise over a period of 2 hours while keeping the temperature of the aqueous solution at 30° C. and the pH at 9. Consequently, a precipitate containing cobalt, nickel and manganese was obtained. The precipitate was collected by filtration, washed with water and dried to afford Ni_{0.5}Co_{0.2}Mn_{0.3}(OH)₂. The Ni_{0.5}Co_{0.2}Mn_{0.3}(OH)₂ obtained by the coprecipitation method was calcined at 520° C. for 5 hours while controlling the oxygen concentration to 25 vol %, thus giving Ni_{0.5}Co_{0.2}Mn_{0.3}Oₓ. Next, the oxide was mixed with a prescribed amount of Li₂CO₃, and the mixture was calcined at 870° C. for 12 hours while controlling the oxygen concentration to 25 vol %. Thus, layered Li_{1.08}Ni_{0.50}Co_{0.20}Mn_{0.30}O₂ (lithium transition metal composite oxide) was prepared. The lithium transition metal composite oxide obtained had a void content of 10% and a crystallite size of 49 nm.

### [Fabrication of Positive Electrode]

The lithium transition metal composite oxide described above was used as a positive electrode active material. The active material, acetylene black and polyvinylidene fluoride were mixed together in a mass ratio of 95:2.5:2.5. An appropriate amount of N-methyl-2-pyrrolidone (NMP) was added. A positive electrode mixture slurry was thus prepared. Next, the positive electrode mixture slurry was applied onto both sides of an aluminum foil as a positive electrode current collector. The wet films were dried and rolled with a roller. A positive electrode was thus fabricated in which the positive electrode mixture layers were disposed on both sides of the positive electrode current collector. The packing density of the positive electrode was 3.4 g/cm³.

### [Fabrication of Negative Electrode]

Artificial graphite, carboxymethylcellulose sodium (CMC-Na) and styrene butadiene copolymer (SBR) were mixed together in a mass ratio of 98:1:1 in an aqueous solution to give a negative electrode mixture slurry. Next, the negative electrode mixture slurry was applied uniformly onto both sides of a copper foil as a negative electrode current collector. The wet films were dried and rolled with a roller. A negative electrode was thus obtained in which the negative electrode mixture layers were disposed on both sides of the negative electrode current collector. The packing density of the negative electrode active material in the negative electrode was 1.6 g/cm³.

### [Preparation of Nonaqueous Electrolytic Solution]

Fluoroethylene carbonate (FEC) and methyl 3,3,3-trifluoropropionate were mixed together in a volume ratio of 15:85. Into the mixed solvent, lithium hexafluorophosphate (LiPF₆) was dissolved in a ratio of 1.2 mol/L. A nonaqueous electrolytic solution was thus prepared.

### [Fabrication of Nonaqueous Electrolyte Secondary Battery]

A 18650 cylindrical nonaqueous electrolyte secondary battery having a nominal capacity of 2300 mAh was fabricated using the positive electrode, negative electrode and nonaqueous electrolytic solution described above, and a polyethylene microporous film as a separator.

The nonaqueous electrolyte secondary battery fabricated has a structure illustrated in FIG. 1, and includes a stainless steel battery case and an electrode assembly accommodated in the case. The electrode assembly includes the positive electrode and the negative electrode wound into a coil via the separator. An upper insulating plate and a lower insulating plate are disposed on and under the electrode assembly. The open end of the battery case is crimped together with a sealing plate via a gasket so as to seal the inside of the battery case. An end of a positive electrode lead made of aluminum is attached to the positive electrode, and the other end of the positive electrode lead is welded to the sealing plate which also serves as a positive electrode terminal. An end of a negative electrode lead made of nickel is attached to the negative electrode, and the other end of the negative electrode lead is welded to the battery case which also serves as a negative electrode terminal. FIG. 2 shows a CP sectional SEM image of the positive electrode active material before charge and discharge cycles. FIG. 3 shows a CP sectional SEM image of the positive electrode active material after 400 cycles.

### Comparative Example 1

A nonaqueous electrolyte secondary battery was fabricated in the same manner as in EXAMPLE 1, except that the nonaqueous electrolytic solution was prepared by mixing FEC, ethylene carbonate (EC), propylene carbonate (PC), ethyl methyl carbonate (EMC) and dimethyl carbonate (DMC) in a volume ratio of 10:10:5:45:30 and dissolving lithium hexafluorophosphate (LiPF₆) into the mixed solvent in a ratio of 1.2 mol/L. FIG. 4 shows a CP sectional SEM image of the positive electrode active material after 400 cycles. The cross section of the particles before charge and discharge cycles was similar to that in EXAMPLE 1 (see FIG. 2).

### Comparative Example 2

A lithium transition metal composite oxide (a positive electrode active material) and a nonaqueous electrolyte secondary battery were prepared in the same manner as in EXAMPLE 1, except that in the preparation of the lithium transition metal composite oxide, a precipitate containing cobalt, nickel and manganese was obtained while maintaining the temperature of the aqueous solution at 40° C., and that the mixture of Ni_{0.5}Co_{0.2}Mn_{0.3}Oₓ with a prescribed amount of Li₂CO₃ was calcined at a calcination temperature of 900° C. while controlling the oxygen concentration to 28 vol %. The lithium transition metal composite oxide obtained had a void content of 0.1% and a crystallite size of 57 nm. FIG. 5 shows a CP sectional SEM image of the positive electrode active material before charge and discharge cycles. The cross section of the particles after 400 cycles was similar to that in COMPARATIVE EXAMPLE 1 (see FIG. 4).

### Comparative Example 3

A nonaqueous electrolyte secondary battery was fabricated in the same manner as in EXAMPLE 1, except that the nonaqueous electrolytic solution was replaced by the nonaqueous electrolytic solution prepared in COMPARATIVE EXAMPLE 1, and that the lithium transition metal composite oxide prepared in COMPARATIVE EXAMPLE 2 was used as the positive electrode active material.

### [Measurement of Average Crystallite Size]

The lithium transition metal oxides were analyzed by the following procedures to measure the average crystallite size.

(1) With use of a powder X-ray diffractometer (manufactured by Rigaku Corporation) using CuKα as an X-ray source, an XRD pattern of each of the lithium transition metal oxides was obtained. The XRD patterns obtained showed that the lithium transition metal oxides all had a hexagonal crystal system and were assigned to space group R-3m based on their symmetry.

(2) Ten peaks of Miller indices 100, 110, 111, 200, 210, 211, 220, 221, 310 and 311 were extracted from the X-ray diffraction pattern of an X-ray diffraction standard (National Institute of Standards and Technology (NIST) Standard Reference Materials (SRM) 660b (LaB6)). The integral width β1 was calculated from the integral intensity and the peak height by the Pawley method using split pseudo-Voigt function.

(3) Ten peaks of Miller indices 003, 101, 006, 012, 104, 015, 107, 018, 110 and 113 were extracted from the X-ray diffraction pattern of the measurement sample (the lithium transition metal composite oxide) and were fitted by the Pawley method using split pseudo-Voigt function. The integral width β2 was calculated from the integral intensity and the peak height.

(4) Based on the results, the integral width β assigned to the measurement sample was calculated using Equation (a) below:

Integral width β assigned to measurement sample=β2−β1 (a)

Using the Halder-Wagner method, β2/tan 2θ was plotted against β/(tan θ sin θ). The average crystallite size of the measurement sample was calculated based on the slope of the approximate line.

### [Evaluation of Batteries]

The batteries were each tested under the following conditions to measure the capacity retention at the discharge rates described (the discharge rate retention), and the capacity retention after 400 cycles (the cycle capacity retention).

### (Charging and Discharging Conditions)

The battery was charged at a constant current of 1150 mA [0.5 It] to a battery voltage of 4.1 V, and was further charged at a constant voltage of 4.1 V until the current value reached 46 mA. After a rest of 10 minutes, the battery was discharged at 1150 mA [0.5 It] to a battery voltage of 3.0 V and was rested for 20 minutes. The temperature during the charging and discharging was 25° C.

### (Discharge Rate Retention)

The discharge rate retention was calculated using the following equation.

Discharge rate retention (%)=(Discharge capacity at 4600 mA [2 It]/Discharge capacity at 1150 mA [0.5 It])×100

The discharge capacity at 1150 mA [0.5 It] was measured by performing charging and discharging under the above charging and discharging conditions. The discharge capacity at 4600 mA [2 It] was measured while changing 1150 mA [0.5 It] in the above discharging conditions to 4600 mA [2 It].

### (Cycle Capacity Retention)

Charging and discharging were repeated 400 times under the above charging and discharging conditions. The capacity retention after 400 cycles was calculated using the following equation.

Capacity retention (%)=(Discharge capacity in 400th cycle/Discharge capacity in 1st cycle)×100

TABLE 1

| | | COMP. | COMP. | COMP. |
|---|---|---|---|---|
| | EX. 1 | EX. 1 | EX. 2 | EX. 3 |
| Void content [%] | 10 | 10 | 0.1 | 0.1 |
| FEC/FMP | Present | Absent | Present | Absent |
| Average crystallite size [nm] | 49 | 49 | 57 | 57 |
| Discharge rate retention [%] | 98 | 94 | 84 | 94 |
| 2 lt/0.5 lt | | | | |
| Cycle capacity retention [%] | 98 | 91 | 95 | 94 |
| 400 cycles | | | | |

From the results described in Table 1, the battery of EXAMPLE 1 outperformed the batteries of COMPARATIVE EXAMPLES 1 to 3 in terms of cycle characteristics and discharge rate characteristics by virtue of its containing FEC and FMP in the electrolytic solution and, at the same time, also because of the presence of voids in the active material particles. In the battery of EXAMPLE 1, FEC and FMP in the electrolytic solution forms a quality film which suppresses the side reaction with the electrolytic solution, on the active surface of the positive electrode active material including the insides of the voids during an initial stage of charge and discharge cycles. Further, a distortion produced by a volume change of the active material during charging and discharging is relaxed by the presence of 10% voids within the particles of the positive electrode active material. Although the reasons are not clear, it is probable that in the battery of EXAMPLE 1, FEC and FMP formed, in the voids of the active material, a specific film with high lithium ion conductivity which was distinct from the surface of the active material. The excellent cycle characteristics and discharge rate characteristics obtained above are probably ascribed to these two effects and the consequent success in suppressing the breakage of the active material particles by the swelling and shrinkage of the active material during charging and discharging.

In contrast, the battery of COMPARATIVE EXAMPLE 1, because of the absence of FEC and FMP in the electrolytic solution, suffered a deposition of decomposition products from the electrolytic solution within the voids after repeated cycles of charging and discharging. The deposits inhibited the swelling and shrinkage of the active material during charging and discharging, thus giving rise to a distortion. Consequently, the active material particles probably cracked from the inside thereof and were broken to cause a decrease in electron conductivity and a decrease in capacity retention (see FIG. 4).

In the batteries of COMPARATIVE EXAMPLES 2 and 3, the substantial absence of voids in the particles of the positive electrode active material (see FIG. 5) caused a failure to relax a distortion produced by a volume change of the active material during charging and discharging, thus resulting in the occurrence of breakage of the active material particles. This is probably the reason for the decreased cycle characteristics as compared to EXAMPLE 1. Further, the battery of COMPARATIVE EXAMPLE 2 showed particularly low discharge rate characteristics.

The above results show that the enhancements in cycle characteristics are not obtained simply by forming voids within the positive electrode active material, and the discharge rate characteristics are not enhanced by mixing FEC and FMP as the electrolytic solution alone. That is, the cycle characteristics are drastically enhanced and the discharge rate characteristics are specifically enhanced (unexpected effects are produced) only when the electrolytic solution includes a fluorinated cyclic carbonate and a fluorinated chain carboxylate ester and, at the same time, when a lithium transition metal oxide with a void content of 0.2 to 30% is used as the positive electrode active material.

### REFERENCE SIGNS LIST

10 NONAQUEOUS ELECTROLYTE SECONDARY BATTERY, 11 POSITIVE ELECTRODE, 12 NEGATIVE ELECTRODE, 13 SEPARATOR, 14 ELECTRODE ASSEMBLY, 15 CASE BODY, 16 SEALING BODY, 17, 18 INSULATING PLATES, 19 POSITIVE ELECTRODE LEAD, 20 NEGATIVE ELECTRODE LEAD, 22 FILTER, 22a FILTER OPENING, 23 LOWER VALVE, 24 INSULATING MEMBER, 25 UPPER VALVE, 26 CAP, 26a CAP OPENING, 27 GASKET

1. A nonaqueous electrolyte secondary battery comprising a positive electrode including a lithium transition metal oxide, a negative electrode and a nonaqueous electrolyte,
the lithium transition metal oxide having a content of voids within particles of 0.2 to 30% before first charging,
the nonaqueous electrolyte including a fluorinated cyclic carbonate and a fluorinated chain carboxylate ester.

2. The nonaqueous electrolyte secondary battery according to claim 1, wherein the lithium transition metal oxide is an oxide represented by the general formula LiₐCoₓNi_{y}M_{(1-x-y)}O₂ {0.1≦a≦1.2, 0<x<0.4, 0.3<y<1, 0.3<x+y<1, and M is at least one selected from Mn, Al and Zr}.

3. The nonaqueous electrolyte secondary battery according to claim 1, wherein the average crystallite size of the lithium transition metal oxide is 40 to 140 nm.

4. The nonaqueous electrolyte secondary battery according to claim 1, wherein the fluorinated cyclic carbonate is fluoroethylene carbonate.

5. The nonaqueous electrolyte secondary battery according to claim 1, wherein the fluorinated cyclic carbonate is present in a proportion of 5 to 40 vol % of the solvent in the nonaqueous electrolyte.

6. The nonaqueous electrolyte secondary battery according to claim 1, wherein the fluorinated chain carboxylate ester is methyl fluoropropionate.

7. The nonaqueous electrolyte secondary battery according to claim 1, wherein the fluorinated chain carboxylate ester is present in a proportion of 20 to 90 vol % of the solvent in the nonaqueous electrolyte.
